(19) United States
(12) Patent Application Publication
(10) Pub. No.: US 2022/0051810 A1
(43) Pub. Date: Feb. 17, 2022

(54) UNTACT TREATMENT SYSTEM

(71) Applicant: HICARENET INC., SEOUL (KR)

(72) Inventors: Hong Jin KIM, Seongnam-si (KR); Gwun Il PARK, Seoul (KR); Ma Ry KIM, Seoul (KR)

(73) Assignee: HICARENET INC., SEOUL (KR)

(21) Appl. No.: 17/128,997

(22) Filed: Dec. 21, 2020

(30) Foreign Application Priority Data
Aug. 13, 2020 (KR) 10-2020-0101902

(51) Int. Cl. G16H 80/00; G16H 15/00; G06T 7/00; G10L 15/26; G10L 25/90; G06F 40/10; G06F 40/58

(52) U.S. Cl. CPC G16H 80/00; G16H 15/00; G06T 7/0012; G06F 40/58; G10L 25/90; G06F 40/10; G10L 15/26

(57) ABSTRACT

Provided is an untact treatment system capable of generating image data with sufficient competence of evidence and reducing the burden of a storage space with respect to storing image data of an untact treatment between a doctor and a patient as evidence in preparation of future treatment detail verification or a medical dispute.

### CROSS-REFERENCE TO RELATED APPLICATION

This application is based on and claims priority under 35 U.S.C. § 119 to Korean Patent Application No. 10-2020-0101902, filed on Aug. 13, 2020, in the Korean Intellectual Property Office, the disclosure of which is incorporated by reference herein in its entirety.

### BACKGROUND

### 1. Field

The present disclosure relates to an untact treatment system for performing an untact treatment between a doctor and a patient, and more particularly, to an untact treatment system for reducing the burden of a data storage space by storing image data of an untact treatment in a smaller capacity based on content of the untact treatment, and automatically generating and providing a report related to the untact treatment, a method of generating image data of an untact treatment with a reduced storage capacity, and a method of generating a report of an untact treatment.

### 2. Description of the Related Art

Recently, demands for the spread of untact treatment systems have been rapidly increased due to issues, such as the expansion of virus infection and the collapse of medical systems, and a telemedicine service system is emerging as a solution. Such telemedicine services may include providing of comprehensive medical services, such as monitoring of biometric information of patients, health guides, telemedicine, issuing of medical reports or prescriptions, and monitoring of subsequent treatments.

In the telemedicine, it is necessary to store, in a database of the untact treatment system, corresponding treatment image data and/or treatment records to be used as evidence when it is required to find out who is responsible for a treatment of a doctor during a medical dispute between the doctor and a patient, such as when an undesirable symptom occurs in the patient as a result of the treatment or therapy, or when a there is a problem in communication between the doctor and the patient.

However, high-definition treatment image data of several to tens of minutes using the untact treatment system significantly increases a required storage capacity compared to an existing simple treatment record, and in particular, to store and maintain all treatment images for all treated patients, it is required to continuously and rapidly expand a storage space of the database of the system and moreover, the burden on database maintenance and management costs increases. On the other hand, when the treatment image data is all stored in low quality, the treatment image data may not be suitable as the evidence because the quality and voice information of a screen are not sufficiently clear to be used as evidence for verifying treatment situations and content when a problem occurs due to a medical dispute or inconsistency in memories on the treatment between the doctor and the patient. Accordingly, there is a need for a method of storing and maintaining untact treatment image data between the doctor and the patient in the database with a small capacity while the untact treatment image data being image data with sufficient competence of evidence.

Also, in a face-to-face treatment, it is common to separately prepare a medical certificate or other medical reports about the patient based on the doctors treatment memory and memo after the treatment. Also, in the untact treatment, there is inconvenience of checking a corresponding image from the beginning to the end to verify explanation and guide a medical staff gave to the patient. Accordingly, in the untact treatment, the image data on the untact treatment between the doctor and the patient is stored as described above, and active studies on how to use the treatment image data stored as such for purposes other than simple treatment are being proceeded.

### SUMMARY

Provided is an untact treatment system capable of generating image data with sufficient competence of evidence and reducing the burden of a storage space with respect to storing image data of an untact treatment between a doctor and a patient as evidence in preparation of future treatment detail verification or a medical dispute.

Also, provided is an untact treatment system capable of automatically generating a medical certificate, other medical reports, or a prescription, based on content of image data on an untact treatment between a doctor and a patient and/or medical information such as consulting information, biometric information, and past medical records of the patient.

Also, provided is an untact treatment system capable of, when a medical certificate, other medical reports, or a prescription generated when a patient is living abroad or a cooperative medical institution or pharmacy is located abroad is to be submitted to a corresponding cooperative medical institution or pharmacy or is to be used as an auxiliary material for a subsequent treatment, generating a medical certificate, other medical reports, or a prescription automatically translated to a language of a residing country of the patient or a country where the cooperative medical institution or pharmacy is located such as to be smoothly used for cooperative and subsequent treatment.

Additional aspects will be set forth in part in the description which follows and, in part, will be apparent from the description, or may be learned by practice of the presented embodiments of the disclosure.

An untact treatment system for an untact treatment between a doctor and a patient, according to an embodiment of the present disclosure, includes:

a communication unit for transmitting and receiving data, and storing, in a database, first image data generated by recording an untact treatment image between the doctor and the patient;

a voice recognition unit for extracting voice data from the first image data, recognizing voice from the voice data, and converting the voice into text data;

a treatment image analysis unit for analyzing a treatment conversation or treatment situation between the doctor and the patient, based on the first image data and the text data, and distinguishing, as a result of the analysis, between a section including a conversation or situation having an importance equal to or greater than a certain value and a section not including the conversation or situation having the importance equal to or greater than the certain value, from the first image data;

a treatment image edition unit for generating the first image data of the section not including the conversation or situation having the importance equal to or greater than the certain value as low-resolution image data having resolution lower than the first image data of the section including the conversation or situation having the importance equal to or greater than the certain value, storing the low-resolution image data, and generating second image data having a reduced storage capacity with respect to the untact treatment between the doctor and the patient by editing and combining the low-resolution image data with a corresponding section of the first image data; and

the database storing the first image data, the second image data, the voice data, and the text data.

The untact treatment system may further include a report generation unit for generating a medical certificate or medical report, based on the text data of the section including the conversation or situation having the importance equal to or greater than the certain value.

The untact treatment system may further include a language conversion unit for converting a medical certificate or medical report prepared in a first language by the report generation unit into a medical certificate or medical report translated in a second language.

The treatment image analysis unit may recognize a section where a pre-determined word or phrase is found from the text data as the section including the conversation or situation having the importance equal to or greater than the certain value.

The treatment image analysis unit may analyze sound information including tone, vibration, volume, and pitch of the doctor's voice or patient's voice or information about behavior of the doctor or patient from the first image data, and recognize a section where a pre-determined abnormal symptom is found as the section including the conversation or situation having the importance equal to or greater than the certain value.

The low-resolution image data may have resolution equal to or greater than a degree that identities of the doctor and patient are identifiable.

The treatment image analysis unit may analyze the treatment conversation or treatment situation between the doctor and the patient based on the first image data and the text data, and when the conversation or situation having the importance equal to or greater than the certain value is identified from the first image data as the result of the analysis, recognize a section from 30 seconds before a start of the conversation or situation to 30 seconds after an end of the conversation or situation as the section including the conversation or situation having the importance equal to or greater than the certain value.

The report generation unit may generate the medical certificate or medical report based on medical data about the patient including interview data, biometric monitoring data, or past medical data, and the text data corresponding to the section including the conversation or situation having the importance equal to or greater than the certain value.

The first language may be a language of a residing country of the doctor and the second language may be a language of a residing country of the patient or a residing country of a doctor performing cooperative diagnosis on the patient.

A method of generating image data on an untact treatment between a doctor and a patient, according to an embodiment of the present disclosure, includes:

a first image data generation step for storing, in a database, first image data generated by recording an untact treatment image between the doctor and the patient;

a voice recognition step for extracting voice data from the first image data, recognizing voice from the voice data, and converting the voice into text data;

a treatment image analysis step for analyzing a treatment conversation or treatment situation between the doctor and the patient, based on the first image data and the text data, and distinguishing, as a result of the analyzing, between a section including a conversation or situation having an importance equal to or greater than a certain value and a section not including the conversation or situation having the importance equal to or greater than the certain value, from the first image data; and

a second image data generation step for generating the first image data of the section not including the conversation or situation having the importance equal to or greater than the certain value as low-resolution image data having resolution lower than the first image data of the section including the conversation or situation having the importance equal to or greater than the certain value, storing the low-resolution image data, generating second image data having a reduced storage capacity with respect to the untact treatment between the doctor and the patient by editing and combining the low-resolution image data with a corresponding section of the first image data, and storing the second image data in the database.

The treatment image analysis step may further include recognizing a section where a pre-determined word or phrase is found from the text data as the section including the conversation or situation having the importance equal to or greater than the certain value.

the treatment image analysis step may further include analyzing sound information including tone, vibration, volume, and pitch of the doctor's voice or patient's voice or information about behavior of the doctor or patient from the first image data, and recognizing a section where a pre-determined abnormal symptom is found as the section including the conversation or situation having the importance equal to or greater than the certain value.

The second image data generation step may further include storing the second image data in the database and then deleting the first image data stored in the database.

The low-resolution image data may have resolution equal to or greater than a degree that identities of the doctor and patient are identifiable.

The treatment image analysis step may further include analyzing the treatment conversation or treatment situation between the doctor and the patient based on the first image data and the text data, and when the conversation or situation having the importance equal to or greater than the certain value is identified from the first image data as the result of the analyzing, recognizing a section from 30 seconds before a start of the conversation or situation to 30 seconds after an end of the conversation or situation as the section including the conversation or situation having the importance equal to or greater than the certain value.

A method of generating a report on an untact treatment between a doctor and a patient, according to an embodiment of the present disclosure, includes:

a first image data generation step for storing, in a database, first image data generated by recording an untact treatment image between the doctor and the patient;

a voice recognition step for extracting voice data from the first image data, recognizing voice from the voice data, and converting the voice into text data;

a treatment image analysis step for analyzing a treatment conversation or treatment situation between the doctor and the patient, based on the first image data and the text data, and distinguishing, as a result of the analyzing, between a section including a conversation or situation having an importance equal to or greater than a certain value and a section not including the conversation or situation having the importance equal to or greater than the certain value, from the first image data; and

a report generation unit for generating a medical certificate or medical report, based on the text data of the section including the conversation or situation having the importance equal to or greater than the certain value.

The report generation step may further include generating the medical certificate or medical report based on medical data about the patient including interview data, biometric monitoring data, or past medical data, and the text data of the section including the conversation or situation having the importance equal to or greater than the certain value.

The method may further include a language conversion step for converting the medical certificate or medical report generated in the report generation step to a medical certificate or medical report translated in a different language.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram of an untact treatment system performing an untact treatment via a network, according to an embodiment of the present disclosure;

FIG. 2 is a block diagram of a configuration of an untact treatment system, according to an embodiment of the present disclosure;

FIG. 3 is a block diagram of a configuration of a user terminal of a patient accessing an untact treatment system, according to an embodiment of the present disclosure;

FIG. 4 is a flowchart of a method of generating image data with reduced storage capacity for an untact treatment between a doctor and a patient, according to an embodiment of the present disclosure;

FIG. 5 is a flowchart of a method of generating a report on an untact treatment between a doctor and a patient, according to an embodiment of the present disclosure; and

FIG. 6 is a schematic diagram of providing an automatic translation service for a report of an untact treatment, according to an embodiment of the present disclosure.

### DETAILED DESCRIPTION

Hereinafter, the present disclosure will be described with reference to accompanying drawings. However, the present disclosure may be implemented in various different forms and is not limited to embodiments described herein. Also, in the drawings, parts irrelevant to the description are omitted in order to clearly describe the present disclosure, and like reference numerals designate like elements throughout the specification.

Throughout the specification, when a part is “connected to (in contact with or combined to)” another part, the part may not only be “directly connected to” the other part, but may also be “indirectly connected to” the other part with another member in between. In addition, when a part “includes” a certain element, the part may further include another element instead of excluding the other element, unless otherwise stated.

Also, the terms used in the present specification are only used to describe specific embodiments, and are not intended to limit the present disclosure. An expression used in the singular encompasses the expression in the plural, unless it has a clearly different meaning in the context. In the present specification, it is to be understood that terms such as “including” or “having”, etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Hereinafter, exemplary embodiments are presented for understanding of the present disclosure, but the embodiments are only examples and it would be obvious to one of ordinary skill in the art that various changes and modifications are possible within the scope and spirit of the present disclosure. Further, it would be obvious that such changes and modifications belong to the appended claims.

Hereinafter, embodiments of the present disclosure will be described in detail with reference to accompanying drawings.

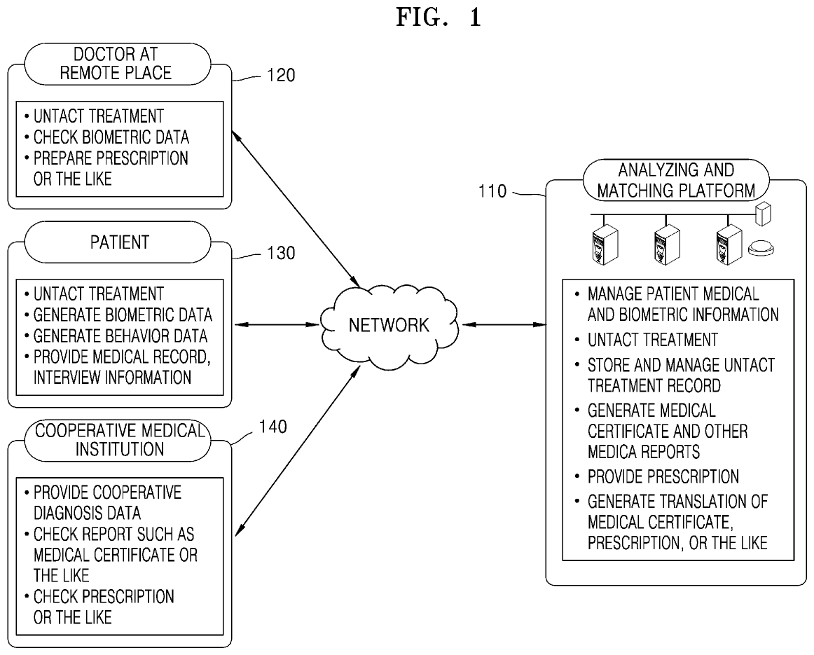

FIG. 1 is a schematic diagram of an untact treatment system 110 according to an embodiment of the present disclosure performing an untact treatment between a doctor 120 and a patient 130 via a network.

Referring to FIG. 1, the doctor 120 or a medical institution at a remote place and the patient 130 may perform an untact treatment or health care by accessing the untact treatment system 110 via the network using respective client terminals. The accessed patient 130 may provide patient medical information, such as a past medical record, biometric data or behavior data generated by a biometric sensor, interview information, and the like, to the untact treatment system 110 for the untact treatment. The untact treatment system 110 may provide the patient medical information pre-provided by the patient 130 to the doctor 120 or medical institution at the remote place, and the doctor 120 or medical institution at the remote place may review the patient medication information, perform a remote untact treatment on the patient 130 via the untact treatment system 110, and prepare a prescription for the patient 130 after the untact treatment.

When there is a cooperative medical institution 140 for the patient 130, the cooperative medical institution 140 may provide cooperative diagnosis-related data to the untact treatment system 110 and receive a medical certificate, a medical report, or a prescription from the untact treatment system 110. When the patient 130 or the cooperative medical institution 140 is located in another country, the untact treatment system 110 may automatically generate and provide a translation of the medical institution, medical report, or prescription in a language used in the other country.

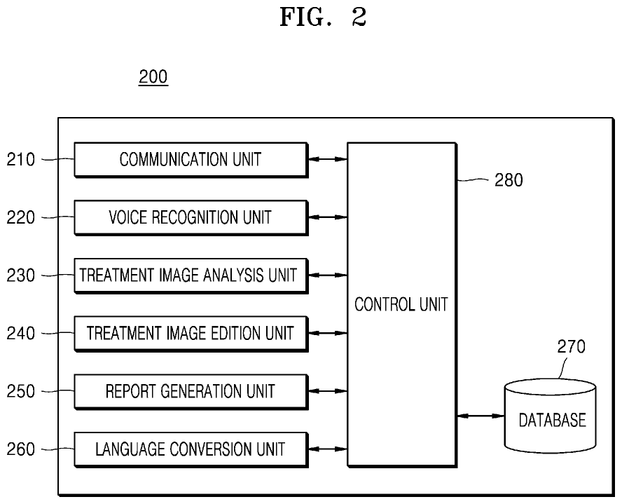

FIG. 2 is a block diagram of a configuration of an untact treatment system 200, according to an embodiment of the present disclosure.

Referring to FIG. 2, the untact treatment system 200 may include a communication unit 210, a voice recognition unit 220, a treatment image analysis unit 230, a treatment image edition unit 240, and a database 270. The untact treatment system 200 may further include a report generation unit 250, a language conversion unit 260, and a control unit 280. The untact treatment system 200 may be implemented in any apparatus capable of large-scale computing, and for example, may be implemented on a separate server or a private or public cloud.

The communication unit 210 may transmit or receive data to or from an external terminal, support a remote image treatment between a doctor and a patient at remote places, generate first image data by recording an image of the remote image treatment between the doctor and the patient, and store the first image data in the database 270. The first image data may have a high resolution of a high definition (HD)-level equal to or greater than 720 p.

The voice recognition unit 220 may extract voice data from the first image data, recognize voice from the voice data, and convert the voice into text data. Voice recognition may be performed via a speech-to-text (SST) and/or voice recognition technique. The voice recognition unit 220 may perform the voice recognition by using a commercial STT application program interface (API). While using the commercial STT API, the voice recognition unit 220 may perform the voice recognition by using an additional voice recognition model to increase accuracy of text conversion. For example, the voice recognition unit 220 may additionally use a medical-specialized voice recognition model trained by being specialized in medical terms and phrases while using the commercial STT API so as to increase accuracy of recognizing conversations in an untact treatment image and converting the conversations into text.

The treatment image analysis unit 230 may analyze a treatment conversation or treatment situation between the doctor and the patient, based on the first image data and/or the text data, and distinguish, as a result of the analysis, between a section including a conversation or situation having importance equal to or greater than a certain value and a section not including the conversation or situation having the importance equal to or greater than the certain value from the first image data. The importance is evaluated by a treatment analysis model based on the treatment conversation or treatment situation between the doctor and the patient, and the certain value of the importance may be a value or level pre-set by the treatment analysis model or a value or level determined via training of the treatment analysis model. However, the certain value of the importance is not limited thereto.

When it is identified that the conversation or situation having the importance equal to or greater than the certain value has occurred considering importance of content of the treatment conversation or a dispute possibility of the treatment situation, the treatment image analysis unit 230 may set a section from 30 seconds before a start of the conversation or situation having the importance equal to or greater than the certain value to 30 seconds after an end of the conversation or situation as the section including the conversation or situation having the importance equal to or greater than the certain value. However, the treatment image analysis unit 230 may set the section by reducing or increasing a time including before and after the conversation or situation depending on the importance of the content of the treatment conversation or a degree of the dispute possibility of the treatment situation. For example, when it is analyzed that the conversation or situation has a high possibility of medical dispute, the importance increases and the time including the before and after the conversation or situation in the section may be increased to 1 minute.

The treatment image analysis unit 230 may determine the importance according to a role-based analysis model based on a pre-input condition. For example, the text data may be analyzed and a section where a pre-determined word or phrase important for treatment content is found may be determined as the section including the conversation or situation having the importance equal to or greater than the certain value. Also, the treatment image analysis unit 230 may determine the importance according to an abnormal symptom analysis model trained via machine learning or deep learning. For example, sound information including tone, vibration, volume, and pitch of the doctor's voice or patient's voice or information about behavior of the doctor or patient may be analyzed from the first image data, and a section where a pre-determined abnormal symptom is analyzed to be present may be determined as the section including the conversation or situation having the importance equal to or greater than the certain value. However, the determining of the conversation or situation having the importance equal to or greater than the certain value may be pre-set or learned according to a treatment analysis model differently from the above.

The treatment image edition unit 240 may generate the first image data of the section not including the conversation or situation having the importance equal to or greater than the certain value as low-resolution image data having resolution lower than the first image data of the section including the conversation or situation having the importance equal to or greater than the certain value, store the low-resolution image data, generate second image data having a significantly reduced storage capacity with respect to the untact treatment between the doctor and the patient by editing and inserting the low-resolution image data to a corresponding section of the first image data, and store the second image data in the database 270. The second image data may be stored in the database 270 by being matched to corresponding text data. The low-resolution image data may have resolution at least equal to or greater than a degree that identities of the doctor and patient are identifiable. The treatment image edition unit 240 may store the second image data in the database 270, and then delete the corresponding first image data from the database 270.

According to such a configuration, the untact treatment system 200 does not need to store entire image data of the remote image treatment between the doctor and the patient in a high resolution that has the burden of storage capacity, but stores, in a high resolution, only an important section that is highly likely to be checked for treatment content or dispute-related evidence later and stores other sections in a low resolution, thereby reducing the burden of electricity, equipment, and costs caused by an increase in a storage space required to maintain treatment image data.

The treatment image edition unit 240 may store the first image data of the section not including the conversation or situation having the importance equal to or greater than the certain value in a low resolution that requires a low storage capacity, store the first image data of the section including the conversation or situation having the importance equal to or greater than the certain value in a high resolution that requires a high storage capacity, and combine images thereof to generate the second image data having a significantly reduced storage capacity compared to the first image data but corresponding to content of the first image data with respect to the untact treatment between the doctor and the patient.

The report generation unit 250 may generate a medical certificate or medical report based on the text data of the section including the conversation or situation having the importance equal to or greater than the certain value. The report generation unit 250 may generate the medical certificate or medical report based on medical information about the patient including interview data, biometric monitoring data, and/or a past medical record, which is received from the patient through the communication unit 210 or stored in the database 270, and/or the text data corresponding to the section including the conversation or situation having the importance equal to or greater than the certain value. However, basic materials for generating a report are not limited thereto.

When the patient or a cooperative medical institution is located in a foreign country, the language conversion unit 260 may automatically translate and provide the medical certificate or medical report generated by the report generation unit 250 in a different language, such as a language of a corresponding country.

The database 270 may store data related to the untact treatment, including the first image data, the second image data, the voice data, and the text data. The database 270 may include management information for the doctor, the patient, or the cooperative medical institution at a remote place. The database 270 may include patient medical information including a past medical record, interview information, biometric and behavior data, a biometric monitoring result, a medical certificate, other medical reports, and a prescription of the patient. The database 270 may include the voice recognition model, the treatment analysis model, and/or data for training and updating such models. The database 270 may include a medical certificate format, other medical report formats, a prescription format, a text data analysis and report generation model, and/or data for training and updating the report generation model. The database 270 may include an automatic translation model for language conversation and related language data. However, data stored in the database 270 is not limited thereto. ( )

The control unit 280 may control the communication unit 210, the voice recognition unit 220, the treatment image analysis unit 230, the treatment image edition unit 240, the report generation unit 250, the language conversion unit 260, and the database 270 such that the untact treatment system 200 provides the untact treatment and/or healthcare service suitable to the patient or the doctor at the remote place. The voice recognition, treatment analysis, and language conversation may be performed by artificial intelligence AI, such as machine learning or deep learning.

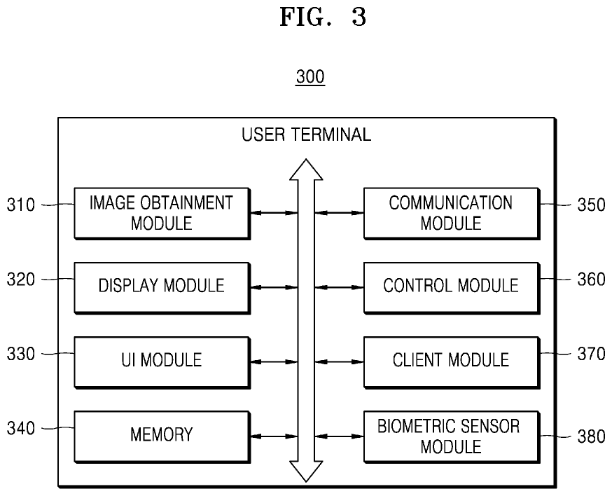

FIG. 3 is a block diagram of a configuration of a user terminal 300 of a patient accessing the untact treatment system 200, according to an embodiment of the present disclosure.

Referring to FIG. 3, the user terminal 300 of the patient may include an image obtainment module 310, a display module 320, a user interface (UI) module 330, a memory 340, a communication module 350, and a control module 360. A user terminal (not shown) of a doctor at a remote place may be similarly configured to include an image obtainment module, a display module, a UI module, a memory, a communication module, and a control module. The user terminal of the doctor and/or the user terminal 300 of the patient may further include a client module 370 downloaded from a cloud or a server where the untact treatment system 200 is implemented. The client module 370 on the user terminal 300 of the patient may further include a biometric sensor module 380. The user terminal 300 of the patient may be an untact treatment-exclusive terminal device, such as Hicare Hub. The user terminal of the doctor at the remote place, the patient, and a cooperative medical institution may be a smart phone, a table computer, a personal computer (PC), a laptop computer, or another computing apparatus where an untact treatment-exclusive application operates.

The image obtainment module 310 may obtain an image of the patient, and for example, may be a camera attached to the outside of the user terminal 300 or included in the user terminal 300. The display module 320 may display a UI for the untact treatment system 200 when the user terminal 300 accesses a server or cloud of the untact treatment system 200. The UI module 330 is a module capable of receiving a user input, and may receive an input via a keyboard, a touchscreen, voice, or the like. The memory 340 may store data required for the user terminal 300 to receive an untact treatment or healthcare service by accessing the server or cloud of the untact treatment system 200. The communication module 350 may enable the user terminal 300 to transmit or receive data to or from the server or cloud of the untact treatment system 200. The control module 360 may control modules in the user terminal 300 to suitably receive the untact treatment or healthcare service.

When the untact treatment system 200 operates in a client-server manner, the user terminal 300 may download and install the corresponding client module 370 by accessing the server or cloud of the untact treatment system 200. The client module 370 may transmit or receive data for providing the treatment and healthcare service to or from the untact treatment system 200, and implement a UI of the untact treatment system 200 on the user terminal 300. The biometric sensor module 380 may directly obtain biometric data of the patient or may obtain, monitor, and/or manage the biometric data of the patient by being connected to a biometric sensor attached to a body of the patient wirelessly or via wires.

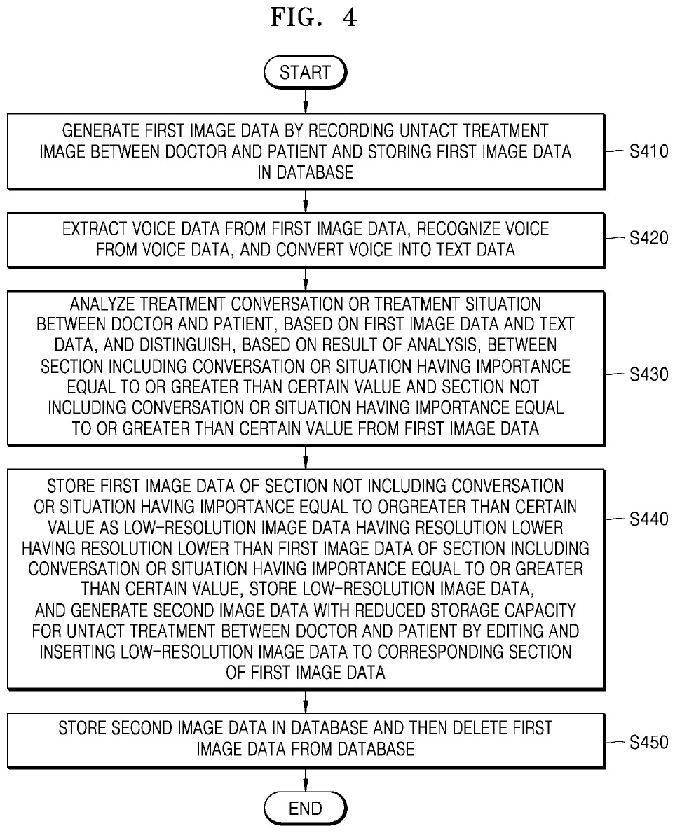

FIG. 4 is a flowchart of a method of generating image data with reduced storage capacity for an untact treatment between a doctor and a patient, according to an embodiment of the present disclosure.

Referring to FIG. 4, the method of generating image data with reduced storage capacity for an untact treatment, according to an embodiment of the present disclosure may include generating first image data by recording an untact treatment image between the doctor and the patient and storing the first image data in the database 270 (operation S410), extracting voice data from the first image data, recognizing voice from the voice data, and converting the voice into text data (operation S420), analyzing a treatment conversation or treatment situation between the doctor and the patient, based on the first image data and the text data, and distinguishing, based on a result of the analysis, between a section including a conversation or situation having importance equal to or greater than a certain value and a section not including the conversation or situation having the importance equal to or greater than the certain value from the first image data (operation S430), and generating the first image data of the section not including the conversation or situation having the importance equal to or greater than the certain value as low-resolution image data having a resolution lower than the first image data of the section including the conversation or situation having the importance equal to or greater than the certain value, storing the low-resolution image data, generating second image data with reduced storage capacity for the untact treatment between the doctor and the patient by editing and combining the low-resolution image data to a corresponding section of the first image data, and storing the second image data in the database 270 (operation S440). The second image data may be stored in the database 270 by being matched to corresponding text data. The low-resolution image data may have resolution at least equal to or greater than a degree that identities of the doctor and patient are identifiable.

The method according to an embodiment of the present disclosure may further include storing the second image data in the database 270 and then deleting the first image data from the database 270 (operation S450).

By using the method according to an embodiment of the present disclosure, the untact treatment system 200 does not need to store entire image data of a remote image treatment between the doctor and the patient in a high resolution that has the burden of storage capacity, but stores, in a high resolution, only an important section that is highly likely to be checked for treatment content or dispute-related evidence later and stores other sections in a low resolution, thereby reducing the burden of electricity, equipment, and costs caused by an increase in a storage space required to maintain treatment image data.

In operation S430, the importance may be determined according to a role-based analysis model based on a pre-input condition. For example, the text data may be analyzed and a section where a pre-determined word or phrase important for treatment content is found may be determined as the section including the conversation or situation having the importance equal to or greater than the certain value. In operation S430, the importance may be determined according to an abnormal symptom analysis model trained via machine learning or deep learning. For example, sound information including tone, vibration, volume, and pitch of the doctor's voice or patient's voice or information about behavior of the doctor or patient may be analyzed from the first image data, and a section where a pre-determined abnormal symptom is analyzed to be present may be determined as the section including the conversation or situation having the importance equal to or greater than the certain value. However, the determining of the conversation or situation having the importance equal to or greater than the certain value may be pre-set or learned according to a treatment analysis model differently from the above.

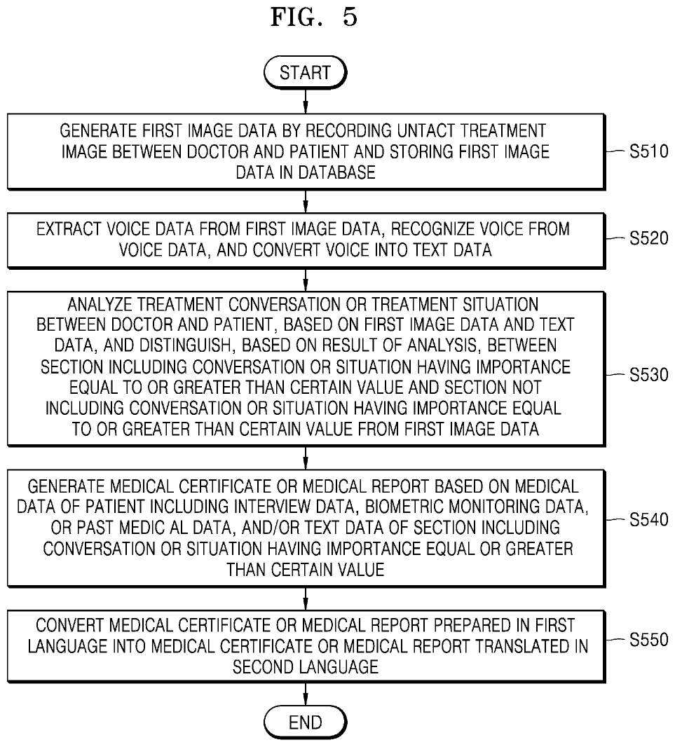

FIG. 5 is a flowchart of a method of generating a report on an untact treatment between a doctor and a patient, according to an embodiment of the present disclosure.

Referring to FIG. 5, the method of generating a report on an untact treatment, according to an embodiment of the present disclosure, may include generating first image data by recording an untact treatment image between the doctor and the patient and storing the first image data in a database (operation S510), extracting voice data from the first image data, recognizing voice from the voice data, and converting the voice into text data (operation S520), analyzing a treatment conversation or treatment situation between the doctor and the patient, based on the first image data and the text data, and distinguishing, based on a result of the analysis, between a section including a conversation or situation having importance equal to or greater than a certain value and a section not including the conversation or situation having the importance equal to or greater than the certain value from the first image data (operation S530), and generating a medical certificate or medical report based on medical data of the patient including interview data, biometric monitoring data, or past medical data, and/or the text data of the section including the conversation or situation having the importance equal to or greater than the certain value (operation S540).

The method according to an embodiment of the present disclosure may further include, when the patient or a cooperative medical institution is located in a foreign country, converting the medical certificate or medical report prepared in a first language into a medical certificate or medical report translated in a second language (operation S550). At this time, the first language may be a language of a residing country of the doctor or a country where an untact treatment system belongs, and the second language may be a language of a residing country of the patient or a residing country of a doctor performing a cooperative diagnosis on the patient.

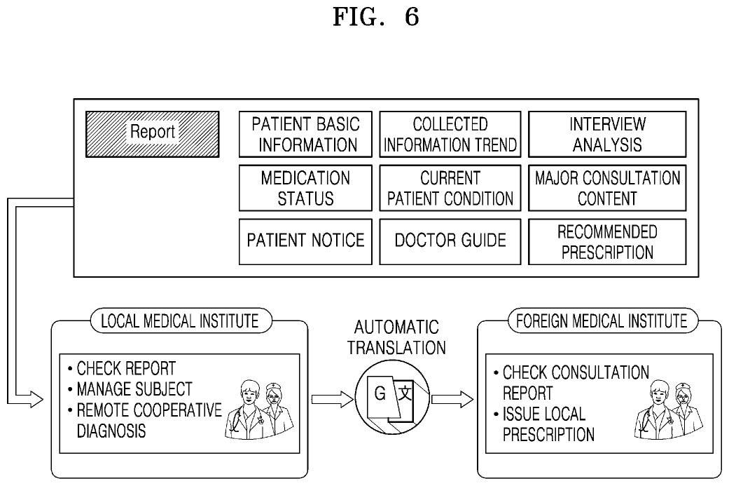

FIG. 6 is a schematic diagram of providing an automatic translation service for a report of an untact treatment, according to an embodiment of the present disclosure.

Referring to FIG. 6, a medical certificate or medical report generated on an untact treatment system may include basic medical information of a patient, a trend of information collected by a biometric sensor or the like, an analysis of an interview obtained from the patient, a medication status of the patient, a current condition of the patient, the content of consultation generated during an untact treatment, a patient's notice, a doctor's guide, and a recommended prescription. Such a medical certificate or medical report may be provided to a doctor or corresponding medical institution that performed the untact treatment to be reviewed or checked by the doctor. Also, when a foreign medical institution performs a function of a cooperative medical institution for the patient, the generated medical certificate or medical report may be automatically translated after a language understandable by the cooperative medical institution is selected or identified on the untact treatment system and then provided to the cooperative medical institution. Through such a configuration, the cooperative medical institution may be able to review the medical certificate or medical report for a treatment or subsequent treatment of the patient, and accordingly, a consecutive and smooth diagnosis or treatment may be enabled for the patient.

The embodiments described above may be implemented as a hardware component, a software component, and/or a combination of the hardware component and the software component. For example, systems, apparatuses, methods, and components described in the embodiments may be implemented by using, for example, one or more general-purpose computers or special purpose computers, such as a processor, a controller, a central processing unit (CPU), a graphics processing unit (GPU), an arithmetic logic unit (ALU), a digital signal processor, a microcomputer, a field programmable gate array (FPGA), a programmable logic unit (PLU), a microprocessor, an application specific integrated circuit (ASIC), a server, or any other device capable of executing and responding to instructions.

Methods according to the embodiments may be recorded on a computer-readable recording medium by being implemented in a form of program commands executed by using various computers. The computer-readable recording medium may include at least one of a program command, a data file, and a data structure. The program commands recorded in the computer-readable recording medium may be specially designed and configured for the embodiments or well known to one of ordinary skill in the computer software field. A hardware device may be configured to operate as one or more software modules to perform the operation of the embodiments, and vice versa.

As described above, although the embodiments have been described by the limited drawings, various modifications and variations are possible from the above description to one of ordinary skill in the art. For example, an appropriate result may be achieved even when the described techniques are performed in a different order from the described method, and/or components of the described systems, structures, devices, and circuits are combined or associated in a form different from the described method or are replaced or substituted by other components or equivalents.

According to an embodiment of the present disclosure, with respect to storing image data of an untact treatment between a doctor and a patient as evidence to prepare for treatment details verification or a medical dispute, which may occur after the untact treatment, the image data may be reconfigured and stored by distinguishing resolutions and qualities according to sections depending on importance of treatment details and/or situations so as to generate image data having sufficient competence of evidence while reducing the burden of a storage space.

Also, according to an embodiment of the present disclosure, by enabling an untact treatment system to automatically generate a medical certificate or other medical reports based on content of image data on an untact treatment between a doctor and a patient and/or medical information of the patient, such as interview information, biometric information, and past medical records, without depending on treatment memories and memos of the doctor, the doctor who performed a treatment may have the low burden of separately preparing the medical certificate and various medical reports and uploading the same in a system and the patient may receive the medical certificate and other medical reports in a short time without having to wait a long time.

Also, according to an embodiment of the present disclosure, by enabling an untact treatment system to automatically translate a medical certificate, other medical reports, or a prescription to a language of a residing country of a patient or a country where a cooperative medical institution or pharmacy is located, the translated medical certificate, other medical reports, or prescription may be provided when the patient is living in a foreign country or the cooperative medical institution or pharmacy used by the patient is located in the foreign country and when the generated medical certificate, other medical reports, or prescription is to be submitted to the cooperative medical institution or pharmacy or to be used as a supplementary material for a subsequent treatment, and thus may be smoothly used for a cooperative diagnosis and subsequent treatment.

The effects of the present disclosure are not limited thereto and should be understood as including all effects that may be inferred from the configuration of the present disclosure in the detailed description or claims.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An untact treatment system for an untact treatment between a doctor and a patient, the untact treatment system comprising:
a communication unit for transmitting and receiving data, and storing, in a database, first image data generated by recording an untact treatment image between the doctor and the patient;
a voice recognition unit for extracting voice data from the first image data, recognizing voice from the voice data, and converting the voice into text data;
a treatment image analysis unit for analyzing a treatment conversation or treatment situation between the doctor and the patient, based on the first image data and the text data, and distinguishing, as a result of the analysis, between a section including a conversation or situation having an importance equal to or greater than a certain value and a section not including the conversation or situation having the importance equal to or greater than the certain value, from the first image data;
a treatment image edition unit for generating the first image data of the section not including the conversation or situation having the importance equal to or greater than the certain value as low-resolution image data having resolution lower than the first image data of the section including the conversation or situation having the importance equal to or greater than the certain value, storing the low-resolution image data, and generating second image data having a reduced storage capacity with respect to the untact treatment between the doctor and the patient by editing and combining the low-resolution image data with a corresponding section of the first image data; and
the database storing the first image data, the second image data, the voice data, and the text data.

2. The untact treatment system of claim 1, further comprising a report generation unit for generating a medical certificate or medical report, based on the text data of the section including the conversation or situation having the importance equal to or greater than the certain value.

3. The untact treatment system of claim 2, further comprising
a language conversion unit for converting a medical certificate or medical report prepared in a first language by the report generation unit into a medical certificate or medical report translated in a second language.

4. The untact treatment system of claim 1, wherein
the treatment image analysis unit recognizes a section where a pre-determined word or phrase is found from the text data as the section including the conversation or situation having the importance equal to or greater than the certain value.

5. The untact treatment system of claim 1, wherein
the treatment image analysis unit analyzes sound information including tone, vibration, volume, and pitch of the doctor's voice or patient's voice or information about behavior of the doctor or patient from the first image data, and recognizes a section where a pre-determined abnormal symptom is found as the section including the conversation or situation having the importance equal to or greater than the certain value.

6. The untact treatment system of claim 1, wherein
the low-resolution image data has resolution equal to or greater than a degree that identities of the doctor and patient are identifiable.

7. The untact treatment system of claim 1, wherein
the treatment image analysis unit analyzes the treatment conversation or treatment situation between the doctor and the patient based on the first image data and the text data, and when the conversation or situation having the importance equal to or greater than the certain value is identified from the first image data as the result of the analysis, recognizes a section from 30 seconds before a start of the conversation or situation to 30 seconds after an end of the conversation or situation as the section including the conversation or situation having the importance equal to or greater than the certain value.

8. The untact treatment system of claim 2, wherein
the report generation unit generates the medical certificate or medical report based on medical data about the patient including interview data, biometric monitoring data, or past medical data, and the text data corresponding to the section including the conversation or situation having the importance equal to or greater than the certain value.

9. The untact treatment system of claim 3, wherein
the first language is a language of a residing country of the doctor and the second language is a language of a residing country of the patient or a residing country of a doctor performing cooperative diagnosis on the patient.

10. A method of generating image data on an untact treatment between a doctor and a patient, the method comprising:
a first image data generation step for storing, in a database, first image data generated by recording an untact treatment image between the doctor and the patient;
a voice recognition step for extracting voice data from the first image data, recognizing voice from the voice data, and converting the voice into text data;
a treatment image analysis step for analyzing a treatment conversation or treatment situation between the doctor and the patient, based on the first image data and the text data, and distinguishing, as a result of the analyzing, between a section including a conversation or situation having an importance equal to or greater than a certain value and a section not including the conversation or situation having the importance equal to or greater than the certain value, from the first image data; and
a second image data generation step for generating the first image data of the section not including the conversation or situation having the importance equal to or greater than the certain value as low-resolution image data having resolution lower than the first image data of the section including the conversation or situation having the importance equal to or greater than the certain value, storing the low-resolution image data, generating second image data having a reduced storage capacity with respect to the untact treatment between the doctor and the patient by editing and combining the low-resolution image data with a corresponding section of the first image data, and storing the second image data in the database.

11. The method of claim 10, wherein the treatment image analysis step further comprises
recognizing a section where a pre-determined word or phrase is found from the text data as the section including the conversation or situation having the importance equal to or greater than the certain value.

12. The method of claim 10, wherein the treatment image analysis step further comprises
analyzing sound information including tone, vibration, volume, and pitch of the doctor's voice or patient's voice or information about behavior of the doctor or patient from the first image data, and recognizing a section where a pre-determined abnormal symptom is found as the section including the conversation or situation having the importance equal to or greater than the certain value.

13. The method of claim 10, wherein the second image data generation step further comprises
storing the second image data in the database and then deleting the first image data stored in the database.

14. The method of claim 10, wherein
the low-resolution image data has resolution equal to or greater than a degree that identities of the doctor and patient are identifiable.

15. The method of claim 10, wherein the treatment image analysis step further comprises
analyzing the treatment conversation or treatment situation between the doctor and the patient based on the first image data and the text data, and when the conversation or situation having the importance equal to or greater than the certain value is identified from the first image data as the result of the analyzing, recognizing a section from 30 seconds before a start of the conversation or situation to 30 seconds after an end of the conversation or situation as the section including the conversation or situation having the importance equal to or greater than the certain value.

16. A method of generating a report on an untact treatment between a doctor and a patient, the method comprising:
a first image data generation step for storing, in a database, first image data generated by recording an untact treatment image between the doctor and the patient;
a voice recognition step for extracting voice data from the first image data, recognizing voice from the voice data, and converting the voice into text data;
a treatment image analysis step for analyzing a treatment conversation or treatment situation between the doctor and the patient, based on the first image data and the text data, and distinguishing, as a result of the analyzing, between a section including a conversation or situation having an importance equal to or greater than a certain value and a section not including the conversation or situation having the importance equal to or greater than the certain value, from the first image data; and
a report generation step for generating a medical certificate or medical report, based on the text data of the section including the conversation or situation having the importance equal to or greater than the certain value.

17. The method of claim 16, wherein the report generation step further comprises
generating the medical certificate or medical report based on medical data about the patient including interview data, biometric monitoring data, or past medical data, and the text data of the section including the conversation or situation having the importance equal to or greater than the certain value.

18. The method of claim 16, further comprising
a language conversion step for converting the medical certificate or medical report generated in the report generation step to a medical certificate or medical report translated in a different language.

19. A computer-readable recording medium having recorded thereon a program for performing the method of claim 10.

20. A computer-readable recording medium having recorded thereon a program for performing the method of claim 16.
